(19) United States
(12) Patent Application Publication
(10) Pub. No.: US 2019/0029930 A1
(43) Pub. Date: Jan. 31, 2019

(54) AQUEOUS COMPOSITIONS AND METHODS FOR IMPROVING THE APPEARANCE OF SKIN

(71) Applicant: L'OREAL, Paris (FR)

(72) Inventors: Yang DENG, Edison, NJ (US); Alexandra Jane Elise FARRAN, Dayton, NJ (US); Anne-Laure Suzanne BERNARD, New York, NY (US); Hy Si BUI, Piscataway, NJ (US); Zhi PAN, Ridgewood, NJ (US)

(21) Appl. No.: 16/047,632

(22) Filed: Jul. 27, 2018

Related U.S. Application Data
(60) Provisional application No. 62/546,656, filed on Aug. 17, 2017.
(60) Provisional application No. 62/539,103, filed on Jul. 31, 2017.

(51) Int. Cl. A61K 8/06; A61Q 19/08; A61K 8/81; A61K 8/31; A61K 8/58

(52) U.S. Cl. CPC A61K 8/06; A61Q 19/08; A61K 8/8117; A61K 2800/43; A61K 8/31; A61K 8/585; A61K 8/8152

(57) ABSTRACT

The disclosure relates to compositions, films, and methods for improving the appearance of the skin. Compositions comprise an oil phase comprising at least one thermoplastic elastomer, at least one adhesive polymer, and a surfactant; an aqueous phase comprising water, a solvent, and a salt; and at least one filler, which together form an association in the composition. Methods comprise applying the compositions to the skin to tighten the skin or hide skin imperfections, by forming a film on the skin.

### TECHNICAL FIELD

The present disclosure relates to cosmetic compositions that provide immediate and long-lasting improvement to the skin. In particular, the compositions provide a physical tightening effect to the skin and are therefore useful for treating eye bags, facial wrinkles, and other age-related skin imperfections.

### BACKGROUND

As Skin produces less collagen and elastin as it ages. For example, after the age of twenty, a person (human) produces about 1 percent less collagen in the skin each year. As a result, the skin becomes thinner and more fragile. Inevitably, wrinkles, crow's feet, age-spots, eye bags, and the like, begin to form.

Consumers often wish to improve the appearance of such age-related skin imperfections, preferably with instantaneous results. Many consumer products and procedures devoted to hiding and reducing wrinkles are available. Some products and procedures are simple and inexpensive, for example, applying make-up, particularly a primer or colored foundation, to cover the skin (and thereby cover and/or fill the wrinkles and provide a smoother look). Far more expensive and drastic procedures, such as surgical face lifts and Botox® injections, are also used to reduce the appearance of wrinkles. However, many consumers either cannot afford, or do not wish, to undergo such drastic cosmetic procedures. There are a number of lotions and creams which are formulated to hydrate the skin and make it more supple, thereby reducing the appearance of wrinkles. Some of these products contain active ingredients, for example, niacinamide, that help repair and rejuvenate skin over time. Unfortunately, however, all of these products and procedures have drawbacks.

Make-up products are often visible, offer minimal texture benefits, and have no long-term lasting effect on the skin. After removal of the make-up, the skin looks the same as before the make-up was applied. Common skin care products can have chronic, acute or both effects on the skin. Hydration and optical effects are common acute benefits, but these benefits quickly wear-off over time.

Attempts have been made to develop new categories of products to improve the appearance of skin without the drawbacks of existing products and procedures. One such family of products can be generally classified as “adhesive, contractile film formers”. Film formers are chemical compositions that when applied to skin, leave a pliable, cohesive and continuous covering. A select group of film formers are also adhesive to the skin and contractile.

### SUMMARY

The disclosure relates to compositions and methods for improving the appearance of the skin. The disclosure provides skin tightening compositions containing water in a water in oil emulsion. Surprisingly, water in the emulsion compositions, maintains a good skin tightening effect and provides freshness, hydration, and easy removal effects.

In one embodiment this disclosure relates to a skin tightening emulsion composition containing:
a) an oil phase comprising: i) at least one thermoplastic elastomer chosen from amorphous hydrocarbon block copolymers of styrene and monomers of hydrocarbon containing 2 to 5 carbon atoms and comprising one or two ethylenic unsaturations, and having a first T_{g} below about 0° C., and a second T_{g} greater than about 25° C.; ii) at least one adhesive film-forming polymer (OD) chosen from polymer particles of C₁-C₄ alkyl(methacrylate)polymer, stablilized in a non-aqueous dispersion; and iii) a surfactant;
b) an aqueous phase comprising: i) water; ii) a solvent; and iii) a salt;
c) at least one filler.

In another embodiment, this disclosure relates to a method for improving the appearance of the skin. The method comprises forming a film on the skin by applying to the skin a skin tightening emulsion composition containing:
a) an oil phase comprising: i) at least one thermoplastic elastomer chosen from amorphous hydrocarbon block copolymers of styrene and monomers of hydrocarbon containing 2 to 5 carbon atoms and comprising one or two ethylenic unsaturations, and having a first T_{g} below about 0° C., and a second T_{g} greater than about 25° C.; ii) at least one adhesive film-forming polymer (OD) chosen from polymer particles of C₁-C₄ alkyl(methacrylate)polymer, stablilized in a non-aqueous dispersion; and iii) a surfactant;
b) an aqueous phase comprising: i) water; ii) a solvent; and iii) a salt;
c) at least one filler;
wherein the film has a Young Modulus greater than about 500 kPa.

### DESCRIPTION OF FIGURES

FIG. 1. DSC measurement of both films. The films were prepared by applying the formulas with a draw-down bar on a Teflon plate with overnight drying on bench.

FIG. 2. FTIR spectrum of both films. The films were prepared by applying the formulas with a draw-down bar on a Teflon plate with overnight drying on bench.

### DETAILED DESCRIPTION

In various embodiments, the disclosure relates to compositions for improving the appearance of the skin. According to various embodiments, the disclosure relates to compositions comprising at least one thermoplastic elastomer, at least one adhesive polymer, and at least one filler.

The compositions may be effective at reducing the appearance of skin imperfections. In various embodiments, the compositions may improve the appearance of the skin by forming a film on the skin that has a Young Modulus greater than that of skin, and thus has the capability of tightening the skin. Additionally, in some embodiments, the film may blur or hide skin imperfections. Accordingly, the disclosure further relates to methods of improving the appearance of the skin by forming a film on the skin with the compositions described herein.

As used herein, the term “long-lasting” means that the film lasts for at least about 6 hours, such as at least about 12 hours, at least about 24 hours, at least about 48 hours, or at least about 72 hours, after the film is formed on the skin.

As used herein, the term “lasting” it is meant to convey that the film is substantially intact in place on the skin.

As used herein, the term “forms quickly” means that the film forms within less than about 20 minutes, such as less than about 15 minutes, or less than about 10 minutes, after the composition is applied to the skin.

As used herein, the term “blur” with regard to skin imperfections means that the visual appearance of the imperfection is less noticeable.

As used herein, the term “tighten” means that the film contracts in a manner that skin has a tighter feel to the user, and that reduces the visual appearance of wrinkles in the skin.

As used herein, the term “soft focus” means that the visual appearance of the skin is more homogenous and matte, leading to the blurring or hiding of skin imperfections.

As used herein, “durable” means the film will not easily rub off, or will not be removed by sweat, water, makeup, lotions, or the like, such that the film will remain substantially intact until removed by the user.

### Compositions

According to various embodiments, the compositions comprise an oil phase comprising at least one thermoplastic elastomer, at least one adhesive polymer, and a surfactant; an aqueous phase comprising water, a solvent, and a salt; and at least one filler, which together form an association in the composition. Additional optional components, such as humectants, and pigments, may also be included in the compositions.

### Thermoplastic Elastomer

According to various embodiments, the at least one thermoplastic elastomer is chosen from amorphous hydrocarbon block copolymers of styrene and monomers of hydrocarbon containing 2 to 5 carbon atoms and comprising one or two ethylenic unsaturations, and having at least two glass transition temperatures (“T_{g}”). The block copolymers may be hydrocarbon-soluble or dispersible in the oily phase.

Non-limiting examples of unsaturated hydrocarbon monomers having 2 to 5 unsaturated carbon atoms include ethylene, propylene, butadiene, isoprene or pentadiene. In various exemplary and non-limiting embodiments, block copolymers may be chosen from those comprising at least one styrene block and at least one block comprising units selected from butadiene, ethylene, propylene, butylene, isoprene, or mixtures thereof. The hydrocarbon-based block copolymer may, for example, be an optionally hydrogenated copolymer comprising styrene blocks and ethylene blocks/C₃-C₄ alkylene or isoprene blocks.

The amorphous hydrocarbon block copolymers comprise at least one first block whose T_{g} is below about 20° C., such as below about 0° C., below about −20° C., or below about −40° C. The T_{g} of the first block can, for example, range from about −150° C. to about 20° C., such as from about −100° C. to about 0° C. The block copolymers also comprise at least one second block whose T_{g} is greater than about 25° C., such as greater than about 50° C., greater than about 75° C., greater than about 100° C., or greater than about 150° C. The T_{g} of the second block can, for example, range from about 25° C. to about 150° C., such as from about 50° C. to about 125° C., about 60° C. to about 120° C., or about 70° C. to about 100° C.

Exemplary, non-limiting amorphous diblock copolymers may be chosen from styrene-ethylene/propylene copolymers, styrene-ethylene/butadiene copolymers, styrene-ethylene/butylene copolymers, styrene-butadiene, or styrene-isoprene copolymers. Diblock copolymers are sold, for example, under the name Kraton® G1701E by Kraton Polymers.

Exemplary, non-limiting amorphous triblock amorphous copolymers may be chosen from styrene-ethylene/propylene-styrene copolymers, styrene-ethylene/butadiene-styrene copolymers, copolymers of styrene-isoprene-styrene, and copolymers of styrene-butadiene-styrene, such as those sold under the names Kraton® G1650, Kraton® D1101, D1102 Kraton®, Kraton® D1160 by Kraton Polymers. In one exemplary embodiment, the thermoplastic elastomer may be a mixture of a triblock copolymer styrene-butylene/ethylene-styrene diblock copolymer and a styrene-ethylene/butylene, such as those sold under the name Kraton® G1657M by Kraton Polymers.

The thermoplastic elastomer may be present in the composition in an amount up to about 10%, such as an amount ranging from about 2% to about 8%, about 4% to about 6%, and can be about 2%, 3%, 4%, 5%, 6%, 7%, or 8%, by weight, for example, relative to the weight of the composition.

### Adhesive Polymer

Compositions according to the disclosure further comprise at least one adhesive film-forming polymer chosen from polymer particles of C₁-C₄ alkyl(methacrylate)polymer, stablilized in a non-aqueous dispersion, referred to herein for ease of reference as an “oil dispersion,” such as those described in WO2015/091513 which is incorporated by reference herein.

By way of example, the C₁-C₄ alkyl (meth)acrylate monomers may be chosen from methyl (meth)acrylate, ethyl (meth)acrylate, n-propyl (meth)acrylate, isopropyl (meth)acrylate, n-butyl (meth)acrylate and tert-butyl (meth)acrylate. For example, the polymer may be a methyl acrylate and/or ethyl acrylate polymer.

The polymer may also comprise an ethylenically unsaturated acid monomer or the anhydride thereof, chosen especially from ethylenically unsaturated acid monomers comprising at least one carboxylic, phosphoric or sulfonic acid function, such as crotonic acid, itaconic acid, fumaric acid, maleic acid, maleic anhydride, styrenesulfonic acid, vinylbenzoic acid, vinylphosphoric acid, acrylic acid, methacrylic acid, acrylamidopropanesulfonic acid or acrylamidoglycolic acid, and salts thereof. For example, the ethylenically unsaturated acid monomer may be chosen from (meth)acrylic acid, maleic acid, and maleic anhydride.

The salts may be chosen from salts of alkali metals, for example sodium or potassium; salts of alkaline-earth metals, for example calcium, magnesium or strontium; metal salts, for example zinc, aluminum, manganese or copper; ammonium salts of formula NH₄⁺; quaternary ammonium salts; salts of organic amines, for instance salts of methylamine, dimethylamine, trimethylamine, triethylamine, ethylamine, 2-hydroxyethylamine, bis(2-hydroxyethyl)amine or tris(2-hydroxyethyl)amine; lysine or arginine salts.

The polymer of the particles of the oil dispersion may thus comprise or consist essentially of about 80% to about 100%, by weight, of C₁-C₄ alkyl (meth)acrylate and of about 0% to about 20%, by weight, of ethylenically unsaturated acid monomer, relative to the total weight of the polymer. According to one exemplary embodiment, the polymer consists essentially of a polymer of one or more C₁-C₄ alkyl (meth)acrylate monomers. According to another exemplary embodiment, the polymer consists essentially of a copolymer of C₁-C₄ (meth)acrylate and of (meth)acrylic acid or maleic anhydride.

By way of non-limiting example only, the polymer of the particles in the oil dispersion, which may optionally be crosslinked or alternatively may not be crosslinked, may be chosen from methyl acrylate homopolymers, ethyl acrylate homopolymers, methyl acrylate/ethyl acrylate copolymers, methyl acrylate/ethyl acrylate/acrylic acid copolymers, methyl acrylate/ethyl acrylate/maleic anhydride copolymers, methyl acrylate/acrylic acid copolymers, ethyl acrylate/acrylic acid copolymers, methyl acrylate/maleic anhydride copolymers, and ethyl acrylate/maleic anhydride copolymers.

The polymer of the particles in the dispersion may have a number-average molecular weight ranging from about 2000 to about 10,000,000, for example ranging from about 150,000 to about 500,000. The polymer particles may be present in the oil dispersion in a content ranging from about 20% to about 60%, for example about 21% to about 58.5%, about 30% to about 50%, about 35% to about 45%, or about 36% to about 42%, by weight, relative to the total weight of the oil dispersion.

The stabilizer in the oil dispersion may be an isobornyl (meth)acrylate polymer chosen from isobornyl (meth)acrylate homopolymer and statistical copolymers of isobornyl (meth)acrylate and of C₁-C₄ alkyl (meth)acrylate present in an isobornyl (meth)acrylate/ C₁-C₄ alkyl (meth)acrylate weight ratio of greater than about 4, for example greater than about 4.5, or greater than about 5. For example, the weight ratio may range from about 4.5 to about 19, such as from about 5 to about 19, or from about 5 to about 12.

By way of example only, the stabilizer may be chosen from isobornyl acrylate homopolymers, statistical copolymers of isobornyl acrylate/methyl acrylate, statistical copolymers of isobornyl acrylate/methyl acrylate/ethyl acrylate, and statistical copolymers of isobornyl methacrylate/methyl acrylate.

In various embodiments, the stabilizer may have a number-average molecular weight ranging from about 10,000 to about 400,000, such as from about 20,000 to about 200,000.

In various embodiments, the combination of the stabilizer+polymer of the particles present in the oil dispersion comprises from about 10% to about 50%, such as about 15% to about 30%, by weight of polymerized isobornyl (meth)acrylate, and from about 50% to about 90%, such as about 70% to about 85%, by weight of polymerized C₁-C₄ alkyl (meth)acrylate, relative to the total weight of the combination of the stabilizer +polymer of the particles.

The oily medium of the oil dispersion comprises a hydrocarbon-based oil. The hydrocarbon-based oil is oil that is liquid at room temperature (25° C.). The term “hydrocarbon-based oil” means oil formed essentially from, or even consisting of, carbon and hydrogen atoms, and optionally oxygen and nitrogen atoms, and not containing any silicon or fluorine atoms. It may contain alcohol, ester, ether, carboxylic acid, amine and/or amide groups.

Exemplary and non-limiting embodiments of the hydrocarbon-based oil medium of the oil dispersion include hydrocarbon-based oils containing up to about 40, such as from 8 to 16 or from 8 to 14, carbon atoms. Optionally, the hydrocarbon-based oil is apolar. For example, the hydrocarbon based oil may be chosen from isododecane.

The oil dispersion may be prepared, for example, as described in

W02015/091513.

In various embodiments, the adhesive polymer may have a T_{g} greater than about 25° C., such as greater than about 50° C., greater than about 75° C., or greater than about 100° C., according to various embodiments.

The at least one adhesive polymer may be present in the composition in an amount up may be present in the composition in an amount up to about 10%, such as an amount ranging from about 2% to about 8%, about 4% to about 6%, and can be about 2%, 3%, 4%, 5%, 6%, 7%, or 8%, by weight, for example, relative to the weight of the composition.

According to various embodiments, the oil phase comprising of the compositions further comprises a surfactant. The surfactant can be chosen from cetyl peg/ppg-10/1 dimethicone, polyglyceryl-4 isostearate, peg-30 dipolyhydroxystearate, cetyl peg/ppg-10/1 dimethicone, polyglyceryl-4 isostearate, Polyglyceryl-3 Pentaolivate, Cyclopentasiloxane (and) PEG/PPG-20/15 Dimethicone, Cyclomethicone (and) PEG/PPG-20/15 Dimethicone, PEG/PPG-20/15 Dimethicone (and) Diisopropyl Adipate, Polyglyceryl-10 Pentaoleate, or the mixtures thereof. The preferred surfactant is the mixture of cetyl peg/ppg-10/1 dimethicone, and polyglyceryl-4 isostearate.

The surfactant may be present in the composition in an amount up may be present in the composition in an amount up to about 10%, such as an amount ranging from about 1% to about 10%, about 4% to about 6%, and can be about 1%, 2%, 3%, 4%, 5%, 6%, 7%, 8%, 9%, or 10% by weight, for example, relative to the weight of the total composition.

Solvents

The oil phase of the compositions may comprise at least one solvent. Optionally, the compositions may comprise at least one solvent chosen from solvents having a vapor pressure at room temperature (25° C.) of greater than about 100 Pa, such as greater than about 500 Pa, or greater than about 1000 Pa. In various embodiments, the composition is free or substantially free of solvents having a vapor pressure at room temperature (25° C.) of less than about 25 Pa. In further embodiments, the composition may comprise at least one solvent having a vapor pressure at room temperature (25° C.) of greater than about 100 Pa, such as greater than 500 Pa, or greater than 1000 Pa, and at least one solvent having a vapor pressure at room temperature (25° C.) of less than about 100 Pa, such as less than about 50 Pa, or less than about 25 Pa.

In various embodiments, the compositions comprise at least one volatile organic solvent. The volatile organic solvent may be chosen from, for example, volatile hydrocarbon-based oils and volatile silicone oils.

For example, volatile hydrocarbon oils include, but are not limited to, those having from 8 to 16 carbon atoms and their mixtures, such as branched C₈ to C₁₆ alkanes and C₈ to C₁₆ isoalkanes (also known as isoparaffins), isododecane, isodecane, isohexadecane. For example, the at least one solvent may be chosen from the oils sold under the trade names of Isobar® or Permethyl®, the C₈ to C₁₆ branched esters such as isohexyl or isodecyl neopentanoate and their mixtures. In at least certain embodiments, the volatile hydrocarbon oils have a flash point of at least 40° C. It is also possible to use mixtures of isoparaffins and other volatile hydrocarbon-based oils, such as petroleum distillates.

Further, volatile silicone oils may be chosen from linear or cyclic silicone oils, such as those having a viscosity at room temperature (25° C.) of less than or equal to 6 cSt and having from 2 to 7 silicon atoms, these silicones being optionally substituted with alkyl or alkoxy groups of 1 to 10 carbon atoms. Examples of volatile silicone oils that may be used include, but are not limited to, octamethyltetrasiloxane, decamethylcyclo-pentasiloxane, dodecamethylcyclohexasiloxane, heptamethyloctyltrisiloxane, hexamethyldisiloxane, decamethyltetrasiloxane, dodecamethylpentasiloxane, and their mixtures. In at least certain embodiments, the volatile silicone oils have a flash point of at least 40° C.

Additionally, the at least one volatile solvent may be chosen from polar volatile solvents, including but are not limited to, alcohols, volatile esters and volatile ethers.

The at least one solvent may be present in oil phase of the compositions in an amount up to about 50%, such as up to about 45%, up to about 40%, up to about 35%, up to about 30%, up to about 25%, up to about 20%, up to about 15%, up to about 10%, or up to about 5%, by the total weight of the composition. For example, the at least one solvent may be present in the composition in an amount ranging from about 5% to about 50%, such as about 15% to about 45%, or about 20% to about 40%, or about 33% to about 36%.

The composition of this disclosure further comprises an aqueous phase comprising water, a solvent, and a salt.

The solvent of the aqueous phase of the composition can be chosen from butylene glycol, ethanol, benzyl alcohol, butylene glycol, ethanol, PEG 4, PEG 8, propylene glycol, dipropylene glycol and eldew (isopropyl lauroyl sarcosinate), or the mixtures thereof.

The salt can be an organic or inorganic salt, it can be chosen from magnesium sulfate, zinc sulfate, sodium EDTA, calcium chloride, potassium chloride, sodium chloride, or the mixtures thereof. Preferably, the salt is a sulfate salt, and most preferably magnesium sulfate.

The solvent can be included in the composition in an amount up to about 25%, up to about 20%, up to about 15%, up to about 13%, for example about 5% to 25%, about 10% to 20% and about 12% to 15%, by weight of the total composition.

The salt can be included in the composition in an amount up to about 5%, up to about 3%, up to about 1%, up to about 0.6%, for example about 0.1% to 5%, about 0.3% to 2% and about 0.5% to 0.7%, by weight of the composition.

Water can be included in the composition in an amount up to about 50%, up to about 45%, up to about 40%, up to about 35%, for example about 20% to 50%, about 25% to 40% and about 30% to 35%, by weight of the composition. The composition is in the form of a water-in-oil (W/O) emulsion.

Additional optional components, such as humectants, and pigments, may also be included in the aqueous phase of the compositions.

### Fillers

The compositions also comprise at least one filler. The fillers may be in the oil phase, aqueous phase or between the two phases of the emulsion composition. The fillers may be mineral or organic in nature, and of any shape. In various embodiments, the fillers may have a particle size greater than about 100 nm, and/or a specific surface area greater than about 200 m²/g.

By way of non-limiting example, fillers may be chosen from talc, mica, silica, silica surface-treated with a hydrophobic agent, fumed silica, kaolin, polyamide (Nylon®) powders (e.g. Orgasol® from Atochem), polyurethane powders, poly-β-alanine powder and polyethylene powder, powders of tetrafluoroethylene polymers (Teflon®), lauroyllysine, starch, boron nitride, hollow polymer microspheres such as those of polyvinylidene chloride/acrylonitrile, for instance Expancel® (Nobel Industrie) or of acrylic acid copolymers (Polytrap® from the company Dow Corning) and silicone resin microbeads (Tospearls® from Toshiba, for example), elastomeric polyorganosiloxane particles, precipitated calcium carbonate, magnesium carbonate, magnesium hydrogen carbonate, hydroxyapatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc stearate, magnesium stearate or lithium stearate, zinc laurate or magnesium myristate.

In at least certain embodiments, the at least one filler may be chosen from hydrophobic silica aerogel particles. Silica aerogels are porous materials obtained by replacing (by drying) the liquid component of a silica gel with air. Hydrophobic silica aerogel particles useful according to embodiments of the disclosure include silylated silica (INCI name: silica silylate) aerogel particles. The preparation of hydrophobic silica aerogel particles that have been surface-modified by silylation is described more fully in U.S. Pat. No. 7,470,725, incorporated by reference herein.

In various embodiments, aerogel particles of hydrophobic silica surface-modified with trimethylsilyl groups may be chosen. For example, the aerogel sold under the name VM-2260® by the company Dow Corning, the particles of which have an average size of about 1000 microns and a specific surface area per unit of mass ranging from 600 to 800 m²/g, or the aerogel sold under the name VM-2270®, also by the company Dow Corning, the particles of which have an average size ranging from 5 to 15 microns and a specific surface area per unit of mass ranging from 600 to 800 m²/g, may be chosen. In other embodiments, the aerogels sold by the company Cabot under the names Aerogel TLD 201®, Aerogel OGD 201®, and Aerogel TLD 203®, CAB-O-SIL TS-530, CAB-O-SIL TS-610, CAB-O-SIL TS-720, Enova Aerogel MT 11008, and Enova Aerogel MT 1200®, may be chosen.

Optionally, mixtures of fillers may be present in the compositions according to the disclosure. For example, a mixture of different aerogel particles, or of an aerogel and a different type of filler, may be used.

The at least one filler may be present in a total amount ranging from about 0.1% to about 10% by weight, for example from about 0.1% to about 5%, from about 0.3% to about 3%, or from about 0.5% to about 1.5%, by weight, relative to the total weight of the composition. In at least certain exemplary embodiments, the filler is present in an amount less than about 3%, such as less than about 2%, by weight, relative to the total weight of the composition. In one embodiment, the filler is present in an amount up to about 1% by weight, relative to the total weight of the composition.

### Additional Components

The compositions according to the disclosure may optionally further comprise additional components, such as humectants and pigments. The additional components may be in the oil phase, aqueous phase or between the two phases of the emulsion composition.

Humectants

Optionally, compositions according to the disclosure may comprise at least one humectant or moisturizing agent. Surprisingly, in at least certain embodiments, the at least one humectant may improve the optical properties and feeling of the film formed on the skin by the composition, without negatively affecting the mechanical properties of the film.

By way of example only, humectants or moisturizing agents may be chosen from polyhydroxy compounds including but not limited to glycerin and glycols such as, for example, propylene glycol, butylene glycol, dipropylene glycol and diethylene glycol, glycol ethers such as monopropylene, dipropylene and tripropylene glycol alkyl(C₁-C₄)ethers, monoethylene, diethylene and triethylene glycol.

The at least one humectant may be present in the composition in an amount up to about 20%, such as up to about 15%, up to about 14%, up to about 13%, up to about 12%, up to about 11%, up to about 10%, up to about 9%, up to about 8%, up to about 7%, up to about 6%, up to about 5%, up to about 4%, up to about 3%, up to about 2%, up to about 1%, or up to about 0.5%, by weight of the composition.

Colorants

The composition may further include at least one colorant, for example to create a colored film on the skin, which may be useful to hide certain skin imperfections. In various embodiments, the at least one colorant may be chosen from dyes, pigments, and nacres.

The at least one colorant may, for example, be chosen from dyes. Non-limiting examples of dyes include Sudan Red, D & C Red 17, D & C Green 6, β-carotene, soybean oil, Sudan Brown, D & C Yellow 11, D & C Violet 2, D & C Orange 5, quinoline yellow and annatto.

In various embodiments, the at least one colorant may be chosen from pigments. As used herein, the term “pigments” is intended to mean white or colored, mineral or organic particles which are insoluble in the composition in which they are present, and which are intended to color and/or opacify the resulting film.

By way of example, inorganic pigments that may be used include titanium oxides, zirconium oxides, cerium oxides, zinc oxides, iron oxides, chromium oxides, ferric blue, manganese violet, ultramarine blue, and chromium hydrate. For example, pigments may be chosen from titanium dioxide and red, black, and/or yellow iron oxide, as well as mixtures thereof.

In further embodiments, pigments with a structure that may be, for example, of silica microspheres containing iron oxide type, may be used. An example of a pigment having this structure is the product sold by the company Miyoshi under the reference PC Ball PC-LL-100 P, constituted of silica microspheres containing yellow iron oxide.

By way of further example, organic pigments that may be used include nitroso, nitro, azo, xanthene, pyrene, quinoline, anthraquinone, triphenylmethane, fluorane, phthalocyanin, metal complex, isoindolinone, isoindoline, quinacridone, perinone, perylene, diketopyrrolopyrrole, indigo, thioindigo, dioxazine, triphenylmethane and quinophthalone compounds. For example, the organic pigments may be chosen from carmine lake, carbon black, aniline black, azo yellow, quinacridone, phthalocyanine blue, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenolic derivatives as described in patent FR 2 679 771.

Nacres may be chosen from white pearlescent pigments such as mica coated with titanium or with bismuth oxychloride, colored pearlescent pigments such as titanium mica with iron oxides, titanium mica with in particular ferric blue or the chromium oxide, titanium mica with an organic pigment of the abovementioned type, and pearlescent pigments based on bismuth oxychloride.

The one or more colorants may optionally be included in the composition in an amount up to about 5%, such as up to about 4.5%, up to about 4%, up to about 3.5%, up to about 3%, up to about 2.5%, up to about 2%, up to about 1.5%, up to about 1%, up to about 0.75%, up to about 0.5%, up to about 0.25%, up to about 0.2%, or up to about 0.1%, by weight, relative to the weight of the composition.

It should be understood that the greater amount of colorant added, the greater the effects of the film on the skin to hide skin imperfections, such as pores, pimples, dark spots, and the like. Therefore, the skilled artisan will be able to choose an amount of colorant appropriate for the composition, keeping in mind the intended use of the final formulation.

### Film

When the compositions according to the disclosure are applied to the skin, the components of the composition together form a matrix that creates a film on the skin. The films formed by the compositions described herein form quickly, are long-lasting and durable, and have optical properties that are advantageous for a skin-tightening film, such as transparency, matte effect, and a soft focus effect which helps to blur skin imperfections so that they are less noticeable.

Additionally, as discussed above, the compositions according to the disclosure form a film that is stiffer than, and thus capable of tightening, human skin. Human skin has a Young Modulus in the range of 10 kPa to 100 kPa, thus, a film for tightening the skin should have a Young Modulus of greater than 100 kPa. The films that are formed by the compositions have Young Modulus' greater than 500 kPa (0.5 MPa) in some embodiments, greater than 1000 kPa (1 MPa) in some embodiments, greater than 5000 kPa (5 MPa) in some embodiments, and even greater than 10,000 kPa (10 MPa) in some embodiments. Additionally, the compositions according to the disclosure have sufficient consistency G* and phase angle below 45°, in order to form an effective and lasting film on the skin.

As such, the amounts and components of the composition should be chosen to provide a film on the skin that is capable of tightening the skin, while also blurring skin imperfections.

In various exemplary embodiments, for the best film properties, it may be advantageous for the total amount of thermoplastic elastomer plus adhesive polymer plus filler to be greater than about 10%, such as greater than about 15% or greater than about 20%, by weight, of the total weight of the composition.

In yet further exemplary embodiments, for the best film properties, it may be advantageous for amounts of the thermoplastic elastomer and adhesive polymer to be chosen so that the ratio of thermoplastic elastomer: adhesive polymer is in the range of about 1:10 to 10:1, in the range of about 1:5 to 5:1, or in the range of about 1:1 to 8:1.

The films may be formed quickly, for example within less than about 30 minutes, less than about 20 minutes, less than about 10 minutes, or less than about 5 minutes, after the composition is applied to the skin.

Films according to the disclosure may be long-lasting. For example, once the composition is applied to the skin and a film is formed, the film may remain substantially intact on the skin for a period of at least about 12 hours, such as at least about 24 hours, at least about 48 hours, or at least about 72 hours.

The films may also be durable. For example, the film may not rub off, may not come off with sweat, or when the film is contacted by water, makeup, lotions, or other products that the user may wish to put on the skin.

### Methods

Methods of improving the appearance of the skin are also disclosed, said methods comprising applying a composition according to the disclosure onto the skin in order to form a film on the skin. Methods comprise tightening the skin, e.g. to get rid of, or reduce the appearance of, wrinkles, eye bags, etc., and/or blurring or hiding skin imperfections, e.g. to camouflage pimples, pores, dark spots, etc.

According to various embodiments, different compositions may be applied to the skin to form films having different properties, such as compositions comprising greater or lesser amounts of pigments depending on whether skin imperfections may require more or less camouflage, etc.

It to be understood that, as used herein the terms “the,” “a,” or “an,” mean “at least one,” and should not be limited to “only one” unless explicitly indicated to the contrary. Thus, for example, reference to “a portion” includes examples having two or more such portions unless the context clearly indicates otherwise.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method that comprises A+B+C include embodiments where a method consists of A+B+C and embodiments where a method consists essentially of A+B+C. As described, the phrase “at least one of A, B, and C” is intended to include “at least one A or at least one B or at least one C,” and is also intended to include “at least one A and at least one B and at least one C.”

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints.

It is understood that when an amount of a component is given, it is intended to signify the amount of the active material.

It should be understood that all patents and published patent applications referenced are incorporated herein in their entireties.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

The compositions and methods according to the present disclosure can comprise, consist of, or consist essentially of the elements and limitations described herein, as well as any additional or optional ingredients, components, or limitations described herein or otherwise known in the art.

It will be apparent to those skilled in the art that various modifications and variations can be made in the delivery system, composition and methods of the invention without departing from the spirit or scope of the invention. Thus, it is intended that the present invention cover the modifications and variations of this invention provided that they come within the scope of the appended claims and their equivalents.

### EXAMPLES

The following Examples are provided for illustrative purposes only, and are not intended to be limiting.

In each of the following examples, the amounts of components given are in terms of active material (AM).

### Dynamic Mechanical Analysis (DMA)

The determination of Young Modulus of the films for all Examples was as follows. The film was made by using a draw down bar at 8″ to cast the solution on a Teflon plate and dried the film at 40° C. in an oven overnight. The DMA Q800FR from TA instruments was used to measure the stress-strain response of the dried film. The deformation was applied from 0% strain to 200% strain at a rate of 100% strain/min at 32° C. Then the Young Modulus of the film was determined from the slope of the stress-strain curve in the linear viscoelastic regime.

### Scanning Electron Microscope (SEM) Measurement

The film sample for SEM was made by using the same method as for DMA measurement. Subsequently, the film was cut into a 5×5 mm piece and loaded onto a stage with a double sided carbon tape. The sample was scanned with a Hitachi TM-1000 Tabletop SEM.

### Rheology Measurement

The rheology of sample solutions was measured by using Rheometer AR-G2 from TA instruments. The dynamic oscillation mode was used with the parallel plate of 20 mm diameter at a gap of 200 μm.

The strain sweep from 0.001% to 1000% at an oscillation frequency of 1 rad/s was applied to the sample at 32° C. The value of elastic modulus G′ and viscous modulus G″ at 10% strain were recorded for each measured sample. The complex modulus G*(consistency) and phase angle El collected at 10% strain (in linear viscoelastic regime) were calculated from the elastic modulus G′ and viscous modulus G″ by the following equations:

$${G}^{″} = \sqrt{{G}^{′2} + {G}^{″2}}$$

$$δ = arctan ( \frac{{G}^{″}}{{G}^{′}} )$$

### Haze and Transparency-BYK Haze-Guard

The film was made by using a draw down bar at 8″ to cast the solution on a transparent plastic film and dried on bench for 3 hours. The BYK Haze-Guard instrument was used to measure the transparency and the haze of the film.

### Gloss—BYK Glossmeter

The film was made by using a draw down bar at 8″ to cast the solution on a transparent plastic film and dried on bench for 3 hours. The BYK Glossmeter was used to measure the gloss and matteness of the film.

### Film Permeability

The film was made by using a draw down bar at 8″ to cast the solution on a Teflon plate and dried the film at 40° C. in an oven overnight. The film was peeled off and cut to 5×5 cm pieces. Each piece was used to cover the top of a scintillation vial filled with 2 mL water, and a piece of Parafilm was used to wrap the piece of film on the side. The weight of each vial was measured immediately as well as different time points. The water weight loss of different films was plotted to the different time points and the evaporation was calculated by fitting the evaporation curve with a linear function. The water vapor permeability of the film (P) is calculated with the followed equation, where (I) is the water vapor permeation flux; (l) is the thickness of the film and the (Δp) is the water vapor pressure difference between the space sealed by the film in the vial and the outside of the film, which is the ambient:

P=I/(Δp/l)

### Contact Angle Measurement

The film was made by using a draw down bar at 8″ to cast the solution on a glass slide and dried on bench overnight. The contact angle of the film on the glass slide was measured by the Biolin Scientific Attension Tensiometer.

### Speed of Drying

The film was made by using a draw down bar at 8″ to cast the solution on a transparent plastic film and weighed regularly during a period of one hour.

### Internal Constraint

The film was made by using a draw down bar at 8″ to cast the solution on a nitrile band and let dry for a period of one hour. As the film shrinks upon drying, the surface of the nitrile band is measured by image analysis.

### Transparency, Homogenizing Power and Whitening Power—Colorimeter MINOLTA

The film was made by casting the solution on a transparent plastic film using a draw down bar (2 mil) and left to dry on the bench for 1 hour. The Minolta colorimeter was used to measure the L, a*, b* and Y of the film, and of a skin tone sheet reference and black and white reference, in order to calculate the transparency, homogenizing power, and whitening power of the films.

### Wear and Coverage

The films were applied on the cheeks of 3 panelists with pimples and pores along nose/cheek area and left for 6 hours. Last was evaluated through before/after pictures. Shininess, pore hiding, imperfections coverage for times both before and after were evaluated.

The following Table 1 shows the compositions of the inventive and comparative formulas and their Young's modulus (MPa) at 32° C., respectively.

TABLE 1
Composition of the inventive and comparative formulas

| | | Comparative | Inventive |
|---|---|---|---|
| Oil phase | CETYL PEG/PPG-10/1 | | 3.0% |
| | DIMETHICONE | | |
| | POLYGLYCERYL-4 | | 1.0% |
| | ISOSTEARATE | | |
| | ISODODECANE | 38.4% | 5.0% |
| | ISOPARAFFIN OIL | 34.3% | 35.0% |
| | KRATON | 11.1% | 5.0% |
| | OD | 9.5% | 5.0% |
| | SILICONE ELASTOMER | 3.8% | |
| Filler | SILICA AEROGEL | 3.0% | 1.0% |
| Water phase | WATER | | 31.6% |
| | BUTYLENE GLYCOL | | 5.5% |
| | MAGNESIUM SULFATE | | 0.6% |
| | ALCOHOL | | 7.3% |

| | | |
|---|---|---|
| Total percentage | 100.0% | 100.0% |
| Young's modulus (MPa) at 32° C. | 20 ± 5 | 6 |

### Procedure for Preparing the Inventive Formulas:

First, 25% Kraton was dispersed in isoparaffin oil with a mechanic stirrer and heated to 90° C. Kept stirring at 90° C. for 1-2 hours until all Kraton polymer was dissolved and polymer solution became clear.

Added the desired amount of Oil dispersion (OD) in isododecane, Kraton/isoparaffin oil solution and free isododecane or isoparaffin oil above with a certain ratio in a container and the mixture was milled with Rayneri at 2000 RPM and 5 min later, predissolved water phase including water, salts, glycol and alcohol was added slowly to the oil phase under milling. The emulsion was milled with Rayneri at 2500RPM for 20 min and then the powder was added under mixing. After all the powder was mixed, the mixture was milled with Rayneri for 10 min.

The final solution was kept at room temperature and sealed to avoid the evaporation of solvents.

Dynamic Mechanical Analysis (DMA)

The film was made by using a draw down bar at 8″ to cast the solution on a teflon plate with the thickness from and dried the film at 40C oven overnight. The DMA Q800FR from TA instruments was used to measure the stress-strain response of the dried film. The deformation was applied from 0% strain to 200% strain at a rate of 100% strain/min at 32° C. Then the Young Modulus of the film was determined from the slope of the stress-strain curve in the linear viscoelastic regime.

DSC Measurement

The DSC measurement of the films was conducted on DSC Q2000 from TA instruments. The film was cooled to −40° C. and then heated to 150° C. with a rate of 10° C./min. The film was made by using a draw down bar at 8″ to cast the formula on a teflon plate and after overnight drying on bench, the film was peeled off for DSC Measurement.

FTIR Measurement

The film was loaded onto STIR card (Thermo Fisher Scientific) for FTIR measurement. The film was made by using a draw down bar at 8″ to cast the formula on a teflon plate and after overnight drying on bench, the film was peeled off for FTIR Measurement.

The resulting inventive formulas were stable emulsion. Compared to the comparative anhydrous formula, the film formed by the inventive formulas had less Young's modulus value (6 MPa, which is 6×10⁶ Pa and 6000 KPa). Therefore, the film was softer and improved the sensorial feel of the film on skin after application. Moreover, the evaporation of the water from inventive formulas after application brought a cooling and fresh feel. In addition, the invention tended to encapsulate water in the film and this film brought a long lasting moisturizing effect.

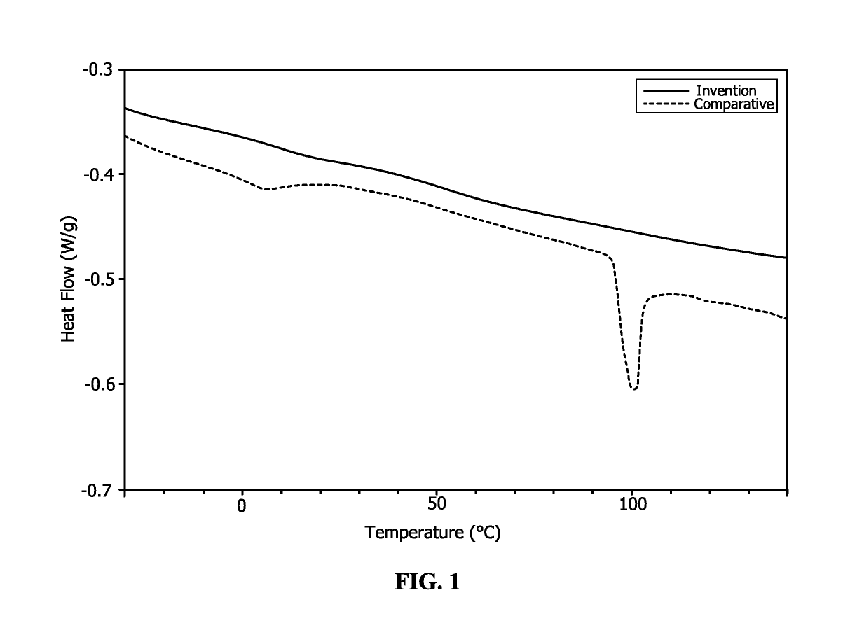

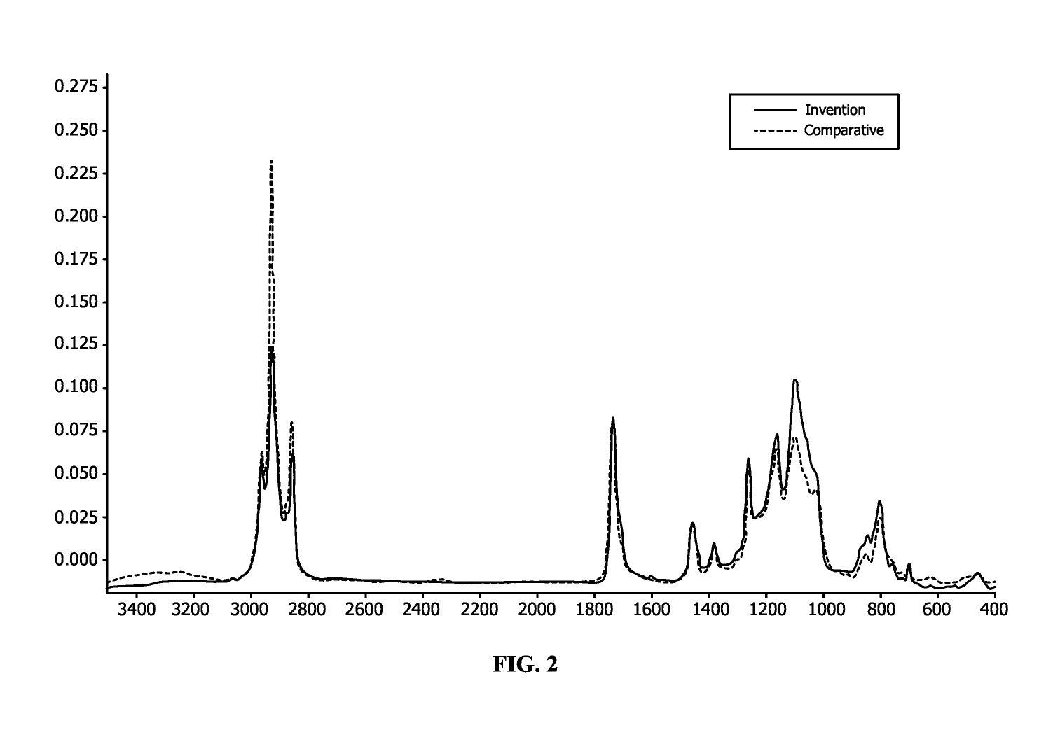

In FIG. 1, after overnight drying on bench, the film formed from invention formula showed a water peak at 100° C. on DSC heating cycle. This water contents was further confirmed by the IR spectrum in FIG. 2. The larger peak of invention at range from 3200-3400 suggested the higher water content in the film.

Formulation with water content ranging from 20% to 50% by weight was the optimal water content in the skin tightening emulsion composition. With low water content, the film was dry and absence of freshness. If the water content was too high the emulsion become unstable and could form a homogenous film.

In conclusion, the inventive skin tightening formulas containing water in the water in oil emulsion maintained a good skin tightening effect and provided freshness, hydration, and easy removal effects.

1. A skin tightening emulsion composition comprising:
a) an oil phase comprising:
i) at least one thermoplastic elastomer chosen from amorphous hydrocarbon block copolymers of styrene and monomers of hydrocarbon containing 2 to 5 carbon atoms and comprising one or two ethylenic unsaturations, and having a first T_{g} below about 0° C., and a second T_{g} greater than about 25° C.;
ii) at least one adhesive film-forming polymer chosen from polymer particles of C₁-C₄ alkyl(methacrylate)polymer, stablilized in a non-aqueous dispersion; and
iii) a surfactant;
b) an aqueous phase comprising:
i) water;
ii) a solvent; and
iii) a salt;
c) at least one filler.

2. The skin tightening composition of claim 1, wherein the at least one thermoplastic elastomer comprises at least one styrene block and at least one block comprising units selected from butadiene, ethylene, propylene, butylene, isoprene, or mixtures thereof.

3. The skin tightening composition of claim 2, wherein the at least one thermoplastic elastomer is chosen from:
diblock copolymers chosen from styrene-ethylene/propylene copolymers, styrene-ethylene/butadiene copolymers, styrene-ethylene/butylene copolymers, styrene-butadiene, or styrene-isoprene copolymers;
triblock copolymers chosen from styrene-ethylene/propylene-styrene copolymers, styrene-ethylene/butadiene-styrene copolymers, copolymers of styrene-isoprene-styrene, or copolymers of styrene-butadiene-styrene; and
mixtures thereof.

4. The skin tightening composition of claim 1, wherein the at least one thermoplastic elastomer is present in the composition in an amount ranging from about 2% to about 10% by weight, relative to the total weight of the composition.

5. The skin tightening composition of claim 1, wherein the at least one adhesive polymer is chosen from polymer particles comprising about 80% to about 100%, by weight, of C₁-C₄ alkyl (meth)acrylate and of about 0% to about 20%, by weight, of ethylenically unsaturated acid monomer of C₁-C₄ alkyl(methacrylate) polymer in an oil dispersion.

6. The skin tightening composition of claim 5, wherein the polymer of the particles is chosen from:
polymers consisting of at one or more C₁-C₄ alkyl(methacrylate)polymer; and
polymers consisting essentially of a copolymer of C₁-C₄ (meth)acrylate and of (meth)acrylic acid or maleic anhydride.

7. The skin tightening composition of claim 5, wherein the C₁-C₄ alkyl(methacrylate)polymer is chosen from methyl(meth)acrylate, ethyl (meth)acrylate, n-propyl (meth)acrylate, isopropyl (meth)acrylate, n-butyl (meth)acrylate and tert-butyl (meth)acrylate polymers.

8. The skin tightening composition of claim 5, wherein the oil comprises hydrocarbon based oils comprising up to 40 carbon atoms.

9. The skin tightening composition of claim 8, wherein the oil is isododecane.

10. The skin tightening composition of claim 5, wherein the polymer particles are stabilized in the oil dispersion by at least one stabilizer chosen from isobornyl acrylate homopolymers, statistical copolymers of isobornyl acrylate/methyl acrylate, statistical copolymers of isobornyl acrylate/methyl acrylate/ethyl acrylate, and statistical copolymers of isobornyl methacrylate/methyl acrylate.

11. The skin tightening composition of claim 1, wherein the at least one adhesive polymer is present in the composition in an amount ranging from about 2% to about 10% by weight, relative to the total weight of the composition.

12. The skin tightening composition of claim 1, wherein the surfactant is chosen from cetyl peg/ppg-10/1 dimethicone, polyglyceryl-4 isostearate, peg-30 dipolyhydroxystearate, cetyl peg/ppg-10/1 dimethicone, polyglyceryl-4 isostearate, Polyglyceryl-3 Pentaolivate, Cyclopentasiloxane (and) PEG/PPG-20/15 Dimethicone, Cyclomethicone (and) PEG/PPG-20/15 Dimethicone, PEG/PPG-20/15 Dimethicone (and) Diisopropyl Adipate, Polyglyceryl-10 Pentaoleate, or the mixtures thereof.

13. The skin tightening composition of claim 12, wherein the surfactant is present in the composition in an amount ranging from about 1% to about 10% by weight, relative to the total weight of the composition.

14. The skin tightening composition of claim 1, wherein the water is present in the composition in an amount from about 20% to about 50% by weight, relative to the total weight of the composition.

15. The aqueous phase of claim 1, wherein the solvent is chosen from butylene glycol, ethanol, benzyl alcohol, butylene glycol, ethanol, PEG 4, PEG 8, propylene glycol, dipropylene glycol and eldew (isopropyl lauroyl sarcosinate), or the mixtures thereof.

16. The aqueous phase of claim 15, wherein the solvent is present in the composition in an amount from about 1% to about 20% by weight, relative to the weight of the composition.

17. The aqueous phase of claim 1, wherein the salt is chosen from magnesium sulfate, zinc sulfate, sodium EDTA, calcium chloride, potassium chloride, sodium chloride, or the mixtures thereof.

18. The aqueous phase of claim 17, wherein the salt is present in the composition in an amount from about 0.1% to about 5% by weight, relative to the weight of the composition.

19. The skin tightening composition of claim 1, wherein the at least one filler is chosen from fillers having a particle size greater than about 100 nm, and/or a specific surface area greater than about 200 m²/g.

20. The skin tightening composition of claim 1, wherein the at least one filler is chosen from silica particles.

21. The skin tightening composition of claim 20, wherein the at least one filler is chosen from hydrophobic silica aerogel particles.

22. The skin tightening composition of claim 21, wherein the at least one filler is chosen from aerogel particles of hydrophobic silica surface-modified with trimethylsilyl groups.

23. The skin tightening composition of claim 19, wherein the aerogel particles is silica silylate particles.

24. The skin tightening composition of claim 1, wherein the at least one filler is present in the composition in an amount ranging from about 0.1% to about 5% by weight, relative to the weight of the composition.

25. The oil phase of the skin tightening composition of claim 1, further comprising at least one solvent chosen from solvents having a vapor pressure at room temperature (25° C.) of greater than about 1000 Pa.

26. The oil phase of claim 25, wherein the at least one solvent is chosen from volatile organic solvents.

27. The oil phase of claim 25, wherein the at least one solvent is chosen from volatile hydrocarbon-based oils and volatile silicone oils.

28. The oil phase of claim 27, wherein the at least one solvent is chosen from branched C₈ to C₁₆ alkanes, C₈ to C₁₆ isoalkanes, isododecane, isodecane, isohexadecane, octamethyltetrasiloxane, decamethylcyclo-pentasiloxane, dodecamethylcyclohexasiloxane, heptamethyloctyltrisiloxane, hexamethyldisiloxane, decamethyltetrasiloxane, dodecamethylpentasiloxane, and mixtures thereof.

29. The skin tightening composition of composition of claim 1, further comprising at least one additional component chosen from, humectants, and colorants.

30. The skin tightening composition of claim 29, comprising at least one humectant chosen from glycerin.

31. The skin tightening composition of claim 29, comprising at least one colorant chosen from organic and inorganic pigments.

32. The skin tightening composition of claim 31, wherein the at least one pigment is present in the composition in an amount ranging up to about 1% by weight, relative to the weight of the composition.

33. The skin tightening composition of claim 1, wherein the at least one thermoplastic elastomer, at least one adhesive polymer, and at least one filler are present in a combined amount of greater than about 10% by weight, relative to the weight of the composition.

34. The skin tightening composition of claim 1, wherein the at least one thermoplastic elastomer, at least one adhesive polymer, and at least one filler are present in a combined amount of greater than about 7% by weight, relative to the weight of the composition.

35. The skin tightening composition of claim 1, wherein the ratio of thermoplastic elastomer:adhesive polymer is in the range of about 1:5 to 5:1.

36. The skin tightening composition of claim 1, wherein the composition has a consistency G* of greater than about 100 Pa (at 10% strain) and a phase angle below about 45°.

37. A skin tightening film formed from the composition of claims 1, wherein the film has a Young Modulus greater than about 500 kPa.

38. The skin tightening film of claim 37, wherein the Young Modulus of the film is greater than about 1000 kPa.

39. A method for improving the appearance of the skin, said method comprising forming a film on the skin by applying a composition according to claim 1 to the skin; wherein the Young Modulus of the film formed on the skin is greater than about 500 kPa.

40. The method of claim 39, wherein the Young Modulus of the film is greater than about 1000 kPa.
